(19) United States
(12) Patent Application Publication
(10) Pub. No.: US 2011/0137164 A1
(43) Pub. Date: Jun. 9, 2011

(54) SYSTEMS AND METHODS TO ACQUIRE AND MANAGE TIMED COUNTS

(76) Inventor: Robin A. Wise, III, San Jose, CA (US)

(21) Appl. No.: 12/962,896

(22) Filed: Dec. 8, 2010

Related U.S. Application Data
(60) Provisional application No. 61/267,723, filed on Dec. 8, 2009.

(51) Int. Cl. A61B 6/00; G06F 17/40

(52) U.S. Cl. 600/436; 702/187

(57) ABSTRACT

A method and a system to acquire and manage timed counts are provided. The timed counts are captured by a gamma probe. For example, a system to acquire the timed counts may comprise a timed counts receiver to receive timed counts during a session of a medical procedure in which the timed counts are captured. A database stores the timed counts in memory during the session of the medical procedure. The database is accessible to generate a user interface during the session or a report based on the stored timed counts.

This application claims the priority benefit under 35 U.S.C. 119(e) of U.S. Provisional Application No. 61/267,723, filed Dec. 8, 2009 and entitled “GALAXY USER INTERFACE,” which is incorporated herein by reference in its entirety.

### TECHNICAL FIELD

The present application relates generally to the technical field of radiation flux measurement systems and, in one specific example, to systems and methods to acquire and manage timed counts.

### BACKGROUND

Hand-held non-imaging scintillation detectors are used for a number of applications in industry and medicine. These applications range from contamination monitoring, used to detect radioactive contamination on surfaces or personnel, to use on humans during operations to guide surgeons to areas of high radiopharmaceutical uptake. The high radiopharmaceutical uptake may indicate tumor activity, as in radio-labeled monoclonal antibody procedures or in a particular organ (e.g., parathyroid or lymph node). To identify tumor activity, intraoperative gamma probes are used in a surgical staging technique known as sentinel node biopsy. This is a method of guiding a surgeon to a lymph node, or nodes, which drains a tumor.

A number of intraoperative gamma probes are in use for producing data to measure local gamma radiation flux in a patient and allow the operator to capture timed counts. The timed counts indicate an amount of radiation released over a period of time (e.g., ten seconds). The timed counts are often presented in the form of an audible tone or a transient display of a numerical value.

Timed counts provide greater information about a location of interest (e.g., a tumor). In practice, a surgeon performs a scan in real-time until a “hot spot” (site of increased activity) is found and then holds the probe in place for a short period—usually 10 seconds—to perform a timed count. Because the amount of radiation detected often varies widely from second to second (due to both the random nature of radiation emission and the random nature of scattering through the body tissues), the longer an operator can hold a probe in place, the more significant the data become. Timed counts are performed to clinically verify the amount of radioactivity in a sentinel node or tumor in a sentinel node biopsy and in procedures such as primary tumor localization using positron emission tomography (PET) and parathyroid localization.

During the sentinel node biopsy, a surgeon may remove one or more nodes based on a “percent primary.” The percent primary represents the ratio of a given count to the “hot spot” count with the greatest activity in that region (the primary). This ratio is represented as a percentage. Typically, a surgeon may remove adjacent nodes exhibiting a percent primary of at least 90% (or some other percentage).

### SUMMARY

A method and a system to acquire and manage timed counts are provided. The timed counts are captured by a gamma probe. For example, a system to acquire the timed counts may comprise a timed counts receiver to receive timed counts during a session of a medical procedure in which the timed counts are captured. The timed counts are stored in a memory during the session of the medical procedure. The database is accessible to generate a user interface during the session or a report based on the stored timed counts.

### BRIEF DESCRIPTION OF THE DRAWINGS

Some embodiments are illustrated by way of example and not limitation in the figures of the accompanying drawings in which:

FIG. 1 is a block diagram depicting an example environment in which some embodiments may be implemented.

FIG. 2 is a block diagram of an example data management system, according to some embodiments.

FIG. 3 is a flowchart of an example method to acquire timed counts, according to some embodiments.

FIG. 4 depicts at least a portion of an example user interface generated during the acquisition of timed counts, according to some embodiments.

FIG. 5 is a diagrammatic representation of machine in the example form of a computer system within which a set of instructions, for causing the machine to perform any one or more of the methodologies discussed herein, may be executed.

### DETAILED DESCRIPTION

Example methods and systems to acquire and manage timed counts are described. In the following description, for purposes of explanation, numerous specific details are set forth in order to provide a thorough understanding of example embodiments. It will be evident, however, to one skilled in the art that the present invention may be practiced without these specific details.

During a session of a medical procedure using an intraoperative gamma probe, a surgeon positions a gamma probe at or near the surface of an object (e.g., a lymph node, tumor, or other tissue) to measure the radioactivity of the object due to radiopharmaceutical uptake. When a surgeon performs a timed count by holding the probe in place for at least a pre-defined period of time, a more accurate measure of radioactivity can be captured. The systems and methods described herein not only display the timed counts to the user (e.g., a surgeon, a nurse, or other medical personnel) but also store the timed counts and related information to allow the user to later retrieve the stored timed counts and related information. The timed counts may be automatically recorded or logged in real time during a session of a medical procedure.

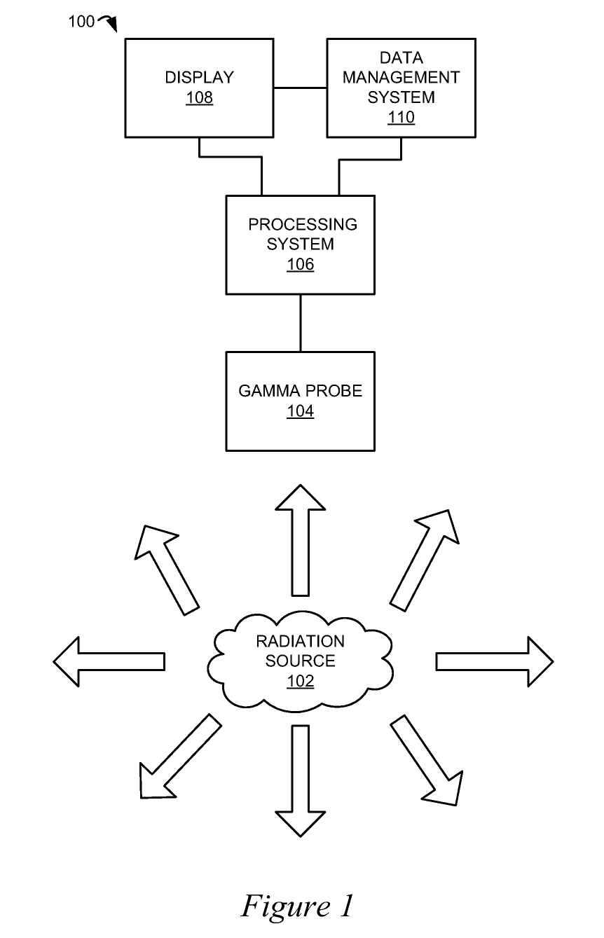

FIG. 1 is a block diagram depicting an example environment 100 in which some embodiments may be implemented. A medical procedure, such as a sentinel node biopsy, may be performed within the environment 100. A sentinel node is the first lymph node (or nodes) to which cancer is likely to spread from a primary tumor. Cancer cells may appear in the sentinel node before spreading (metastasizing) to other lymph nodes. In a sentinel node biopsy, one or more lymph nodes (the sentinel node or nodes) are removed. To identify the sentinel lymph node(s), the surgeon injects a radiopharmaceutical near the tumor. The surgeon uses a scanner (e.g. a gamma probe 104) to identify the sentinel lymph nodes(s) containing the radioactive substance, a radiation source 102. Once the sentinel node is located, the surgeon removes the lymph node(s).

The gamma probe 104 is a hand-held device, used with a Geiger-Muller tube or scintillation counter, for intraoperative use after injection of a radiopharmaceutical into a patient. The gamma probe 102 is used to identify a lymph node as being a sentinel lymph node based on the radiation uptake of that lymph node (e.g., the radiation source 102). The gamma probe 102 may include a button or trigger that, when activated by a surgeon, transmits a signal to initiate a timed count where the number of counts indicative of radiation emitted by the source 102 are counted over a longer period of time (e.g., ten seconds). An example of a gamma probe 104 is included with the C-TRAK® gamma probe by Care Wise Medical of Morgan Hill, Calif.

The processing system 106 is to receive the data collected by the gamma probe. The processing system 106 may process the data to remove noise, artifacts, or the like. The processing system 106, in cooperation with the gamma probe 104, may perform a timed count. In some instances, the processing system 106 may be used in conjunction with a positioning system (not shown) to determine a location of the gamma probe 104 or sentinel node. The processing system 106 further generates a user interface based on the collected data.

The gamma probe 104 in conjunction with the processing system 106 allows the user to take timed counts for a user-defined time period. The term, “timed counts” is used to refer to the total number of counts captured over the user-defined time period, usually ten seconds. A default time period may be set at, for example ten seconds. Alternatively, a shorter or longer time period (or default time period) may be set, ranging from, for example, one second to sixty seconds or longer (e.g., 199 seconds). A plurality of timed counts may be captured over the course of a single session.

The display 108 displays the user interface generated by the processing system 106. The user interface may include a number of counts per second, a timed count displayed for only a short period of time but not stored, and settings such as radiopharmaceutical isotope being used, a threshold for detection, a window of detection, system configurations, and a count time. The display 108 may include one or more speakers that emit audible tones to indicate the detected level of radioactivity.

The data management system 110 is used to acquire and manage timed counts. The data management system 110 receives the timed counts via the processing system 106 or the gamma probe 104 as the timed counts are captured and stores the timed counts into a memory for later retrieval. In some instances, the data management system 110 generates an additional interactive user interface that may be displayed to the user by the display 108 during the session.

In some instances, the data management system 110 may collect additional information about the timed counts during a session. For example, each timed count may be numbered and associated with a timestamp and duration. A user may provide additional information about each timed count such as a location identifier indicating the position of the gamma probe 104 during a timed count and a type identifier indicating a type of tissue associated with the timed count. Additionally, the data management system 110 may perform one or more calculations based on the timed counts and their associated additional information, such as calculating a number of counts per second and calculating a percent primary.

In some embodiments, the display 108 (or an additional display) may include one or more interfaces for receiving and displaying timed counts and additional information during the session or once the session is completed. During a session, the display 108 may switch between one or more interfaces depending on whether a timed count is being performed or has been performed. To illustrate, the display 108 may display an interface as provided by the processing system 106 before a user-initiated timed count and while the timed count is captured. The display 108 may switch to an interface provided by the data management system 110 after the timed count is captured for a pre-defined period of time (e.g., twelve seconds) or until a selection to return to the former interface is received from the user. While active, the interface provided by the data management system 110 may receive and display additional information from the user (e.g., location and type information) and to display calculated values (e.g., counts per second and percent primary) to the user.

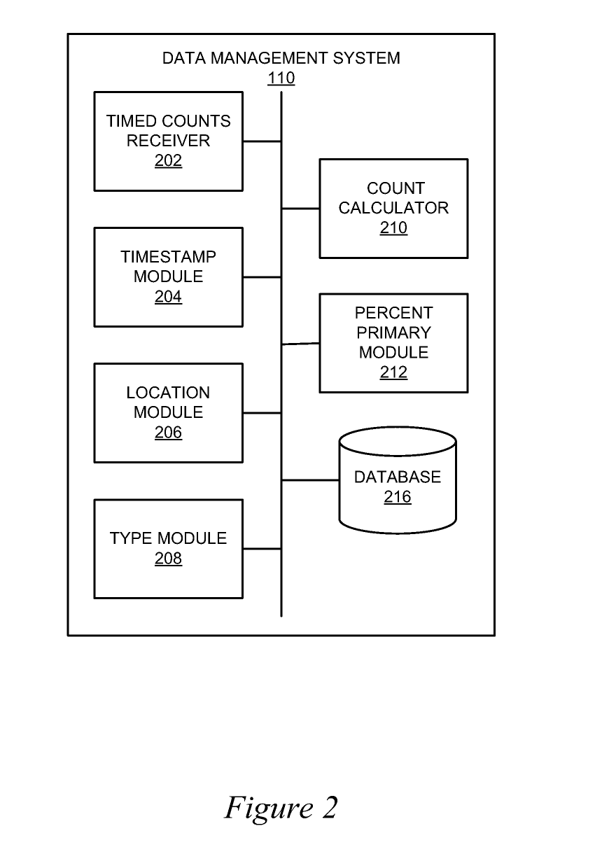

FIG. 2 is a block diagram of an example data management system 110, according to some embodiments. The data management system 108 comprises one or more modules that may be implemented in hardware, software, or a combination of the two.

The timed counts receiver 202 is in electronic communication with the gamma probe 104 and the processing system 106 to capture and store the timed counts in a memory. The timed counts receiver 202 may, in some instances, communicate directly with the gamma probe 104 and capture the timed count substantially in parallel with the processing system 106 or may receive the timed count from the processing system 106 once the timed count is completed. The timed counts receiver 202 may assign a sample number (or other unique identifier) to each timed count.

The timed counts captured during a session may automatically be stored every time a new count is taken. The timed counts themselves may be manually triggered. The automatic storage of the timed counts may include only the most recent data for timed counts (e.g., counts captured within an ongoing session of a medical procedure). For example, if the equipment is turned off and then restarted, the user may restore the stored timed counts for the particular patient who is the subject of examination, and continue the session.

The timestamp module 204 is configured to capture a timestamp indicating when the timed count was captured. The timestamp may reflect when the user initiates the timed count or the time at which the timed count is completed. The timestamp is associated with the timed count in the memory.

The location module 206 is configured to receive and store the location identifier that may be an indication provided by the user of the location at which the timed count was performed. The user may provide the indication by entering text into a text box. In some embodiments, the user interface may include a menu (e.g., a pop-up or drop-down menu) from which the user may select a location identifier from a predetermined list of location identifiers. Examples of location identifiers that may be selected include, but are not limited to: none, in vivo, in vivo left, in vivo right, ex vivo, ex vivo left, ex vivo right, and, for use in perfusion studies, background, background left, and background right.

The type module 208 is configured to receive and store a type identifier that is an indication from the user of the type of tissue at which the associated timed count was performed. The user may provide the type identifier by entering text into a text box. In some embodiments, the user interface may include a menu (e.g., a pop-up or drop-down menu) from which the user may select a type identifier. Examples of type identifiers include, but are not limited to: none, primary (most active) node, sentinel node, tumor, and other.

The count calculator 210 is configured to calculate a number of counts per second for each stored timed count. The counts per second are calculated by dividing the number of counts in the timed counts by the number of seconds in the duration of the timed count. For example, if a timed count is 4000 counts and has a duration of ten second, the number of counts per second is calculated by dividing 4000 counts by 10 ten seconds, resulting in 400 counts per second.

The percent primary module 212 is configured to calculate a percent primary for each timed count. The percent primary is based on a primary count where the primary count is a maximal or greatest known timed count in the scanned area. The percent primary is calculated by dividing a captured timed count by the primary count and the percent primary is, optionally, expressed as a percentage. During a medical procedure, a surgeon may remove lymph nodes or other tissues where the captured timed count meets or exceeds a threshold value of, for example, 90% (or some other percentage) of the primary count. In some embodiments, the percent primary module 212 may further determine if the percent primary exceeds a predetermined threshold and generate an alert based on the determination.

A database 216 stores the captured, received, and calculated information collected by the data management system 110. The database 216 may be stored in a volatile memory, such as random access memory (RAM), during a session. After a session is complete, the information may be stored in a hard drive comprising non-volatile memory. In some embodiments, in the database 216 is generated using Sequential Query Language (SQL).

The database 216 may be accessible to one or more computer systems. In some instances, various reports may be generated based on information gathered over one or more sessions. The reports may be generated, for example, to evaluate treatment progress or to obtain insurance reimbursement. A report may be included in a patient record. In electronic patient records, a link to a report may be provided. In some instances, the reports may be customized by a user. A generated report may include information about timed counts captured over a series of sessions for a particular patient.

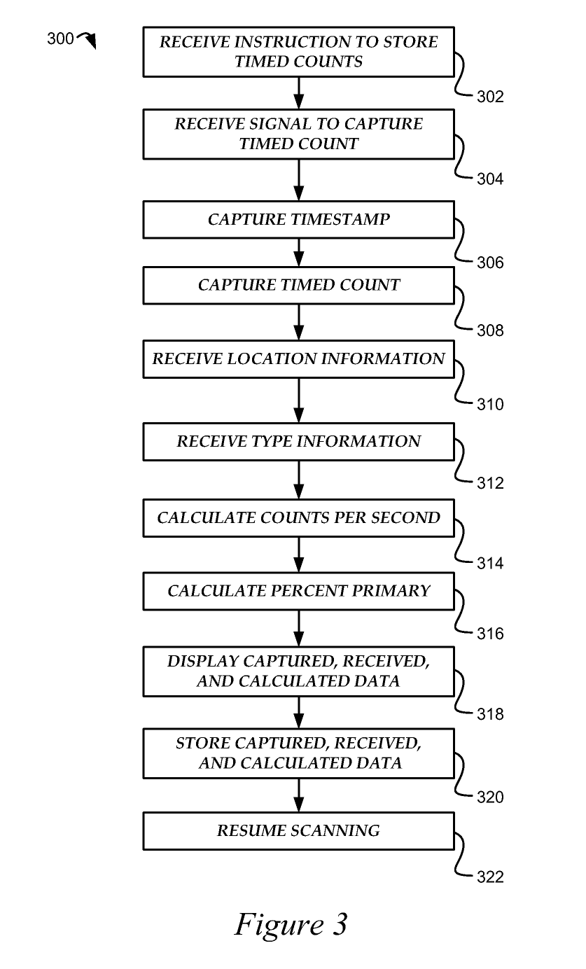

FIG. 3 is a flowchart of an example method 300 to acquire timed counts, according to some embodiments. The method 300 may be performed at least by the data management system 110. It is noted that the order of the operations described may be varied in additional embodiments.

In an operation 302, an instruction to store timed counts is received. In some instances, the instruction to store timed counts is set as a default instruction and may include an indication of the duration of the timed count.

In an operation 304, a signal to capture a timed count is received from, for example, a surgeon. The signal may be generated by pressing a button or by other action. The signal may include a defined duration of the timed count.

In an operation 306, a timestamp is captured. The timestamp may indicate the beginning or the end of the timed count. In an operation 308, the timed count is captured. In some instances, the timed count may be assigned a sample number or other unique identifier.

In an operation 310, a location identifier may be received from a user. In an operation 312, a type identifier may be received from the user. Additional information may also be received from the user in connection with steps 310 and 312.

In an operation 314, the number of counts per second is calculated, and, in an operation 316, the percent primary is calculated. Additional calculations may also be performed, including, for example, comparing the percent primary to a threshold value.

In an operation 318, the captured, received, and calculated data is displayed to the user. In addition, in an operation 320, the captured, received, and calculated data is stored.

In an operation 322, the user may resume scanning to determine an additional location to capture a second timed count.

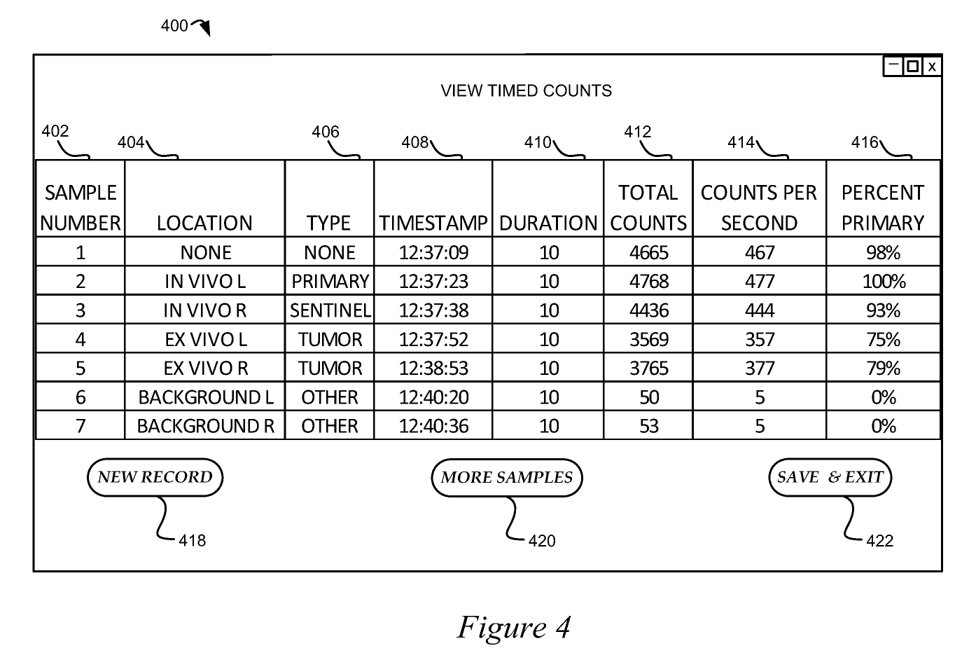

FIG. 4 depicts at least a portion of an example user interface 400 for acquiring timed counts, according to some embodiments. The user interface 400 may be displayed during a session or as part of a report generated after a session.

The interface 400 includes a table having a number of columns. The first column, sample number 402, lists the sample number for each timed count. As depicted, the sample number is assigned sequentially from the beginning of the session. The second column, location 404, displays the location identifier associated with each timed count. The third column, type 406, displays the type identifier associated with each timed count. The fourth column, timestamp 408, displays the timestamp associated with each timed count. The fifth column, duration 410, displays the duration (in seconds) of each timed count. The sixth column, total counts 412, indicates the number of counts taken during each timed count. The seventh column, counts per second 414, indicates the count frequency or calculated number of counts per second for each timed count. The eighth column shown, percent primary 416, indicates the calculated percent primary for each timed count. Thus, for example, sample number 2 in the table of FIG. 4 indicates the greatest known level of radioactivity in the scanned region (having the largest total count for the common scam duration of 10 seconds and having the greatest counts per second value), and therefore provides a primary count with respect to which a percent primary for each respective sample is calculated.

The table of interface 400 may be manipulated by the user in several ways. First, the fields can be customized such that they appear in any order the user wishes, by clicking on a column header and sliding it back and forth. The “sample number” column 402, or another portion of the timed count data tables, may be locked in place on the far left of the data table in the display. The other columns (timestamp 408, location 404, tissue type 406, duration 410, total counts 412, counts per second, and percentage of primary) may be arranged by the user in any order. Additional columns, such as a column to provide an alert that the percent primary threshold is met or exceeded, may be added by the user.

The interface 400 further comprises one or more buttons that provide interaction with the user to receive user input. The new record button 418 opens a new record or retrieves a previously saved record. The more samples button 420 initiates a capture of a next timed count within the same session. The save and exit button 422 saves the current record and exits the current session.

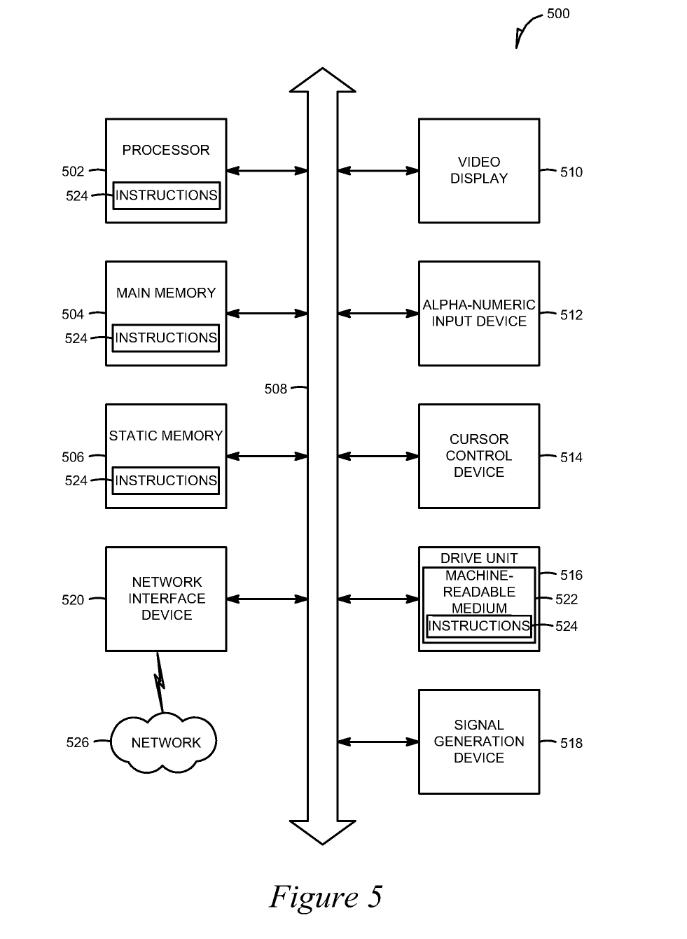

FIG. 5 shows a diagrammatic representation of machine in the example form of a computer system 500 within which a set of instructions, for causing the machine to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 500 includes a processor 502 (e.g., a central processing unit (CPU) a graphics processing unit (GPU) or both), a main memory 504 and a static memory 506, which communicate with each other via a bus 508. The computer system 500 may further include a video display unit 510 (e.g., a liquid crystal display (LCD) or a cathode ray tube (CRT)). The computer system 500 also includes an alphanumeric input device 512 (e.g., a keyboard), a cursor control device 514 (e.g., a mouse), a disk drive unit 516, a signal generation device 518 (e.g., a speaker) and a network interface device 520. The computer system 500 may also include a touchscreen (not shown).

The disk drive unit 516 includes a machine-readable medium 522 on which is stored one or more sets of instructions (e.g., software 524) embodying any one or more of the methodologies or functions described herein. The software 524 may also reside, completely or at least partially, within the main memory 504 and/or within the processor 502 during execution thereof by the computer system 500, the main memory 504 and the processor 502 also constituting machine-readable media.

The software 524 may further be transmitted or received over a network 526 via the network interface device 520.

While the machine-readable medium 522 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. The term “machine-readable medium” shall accordingly be taken to include, but not be limited to, solid-state memories, and optical and magnetic media.

Thus, a method and system to acquire and manage timed counts have been described. Some embodiments may be used to provide a technical solution to the technical problem of recording accurate records and later accessing those records. Although the present invention has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A system comprising:
a timed counts receiver, executed by one or more processors, to receive timed counts captured by a non-imaging radiation sensor during a session of a medical procedure;
a memory to automatically store the timed counts during the session of the medical procedure; and
a data processing module to retrieve the stored timed counts from the memory, and to generate a report based on the stored timed counts.

2. The system of claim 1, wherein the report comprises an interactive user interface generated during the session.

3. The system of claim 1, wherein the report comprises a summary of one or more sessions of the medical procedure.

4. The system of claim 1, wherein the timed counts receiver is further to assign a unique identifier to each of the timed counts at a time each timed count is captured.

5. The system of claim 1, further comprising a location module to receive a location identifier associated with each timed count from the user at a time each timed count is captured.

6. The system of claim 1, further comprising a type module to receive a type identifier associated with each timed count from the user at a time each timed count is captured.

7. The system of claim 1, further comprising a count calculator to calculate a number of counts per second associated with each timed count from the user at a time each timed count is captured.

8. The system of claim 1, further comprising a percent primary module to calculate a percent primary associated with each timed count from the user at a time each timed count is captured.

9. The system of claim 8, wherein the percent primary module is further to compare the percent primary to a pre-defined threshold associated with each timed count at a time each timed count is captured.

10. The system of claim 9, wherein the percent primary module is further to provide an alert to the user if the threshold is met or exceeded at a time each timed count is captured.

11. The system of claim 1, further comprising a timestamp module to record a timestamp associated with each timed count from the user at a time each timed count is captured.

12. A method comprising:
receiving one or more timed counts from a gamma probe during a session of a medical procedure in which the timed counts are captured; and
automatically storing, using one or more processors, the timed counts in a memory during the session of the medical procedure; and
generating a report based on the stored timed counts.

13. The method of claim 12, wherein the report comprises an interactive user interface generated during the session.

14. The method of claim 12, wherein the report comprises a summary of one or more sessions of the medical procedure.

15. The method of claim 12, further comprising assigning a unique identifier to each of the timed counts at a time each timed count is captured.

16. The method of claim 12, further comprising calculating a number of counts per second associated with each timed count from the user at a time each timed count is captured.

17. The method of claim 12, further comprising calculating a percent primary associated with each timed count from the user at a time each timed count is captured.

18. The method of claim 17, further comprising comparing the percent primary to a pre-defined threshold associated with each timed count at a time each timed count is captured.

19. The system of claim 18, further comprising providing an alert to the user if the threshold is met or exceeded at a time each timed count is captured.

20. A non-transitory computer-readable storage medium having instructions embodied thereon, the instructions executable by one or more processors for performing a method for acquiring and managing timed counts, the method comprising:
receiving one or more timed counts from a gamma probe during a session of a medical procedure in which the timed counts are captured; and
automatically storing, using one or more processors, the timed counts in a memory during the session of the medical procedure; and
generating a report based on the stored timed counts.
